(19) United States
(12) Patent Application Publication
(10) Pub. No.: US 2024/0277956 A1
(43) Pub. Date: Aug. 22, 2024

(54) NASAL DELIVERY SYSTEM

(71) Applicant: Suzhou Huafan Chuangshuo Medical Technology Co., Ltd, Pudong (CN)

(72) Inventors: Xu Wen Ng, Singapore (SG); Ty Tiefeng Hu, Pudong (CN)

(73) Assignee: Suzhou Huafan Chuangshuo Medical Technology Co., Ltd, Pudong (CN)

(21) Appl. No.: 18/112,360

(22) Filed: Feb. 21, 2023

(51) Int. Cl. A61M 15/08; A61M 15/00

(52) U.S. Cl. CPC A61M 15/085; A61M 15/0001

(57) ABSTRACT

A device for providing an inhibition agent to a nasal cavity of a user, the device comprising: an insertion element, said insertion element having a surface having the inhibition agent, said insertion element arranged to insert the surface into the nasal cavity, such that the inhibition agent is placed to receive an inhalation and/or exhalation airflow.

### BACKGROUND OF THE INVENTION

### 1. Field of the Invention

The invention relates to delivery systems for delivering an inhibition agent to the nasal cavity.

### 2. Prior Art

The spread of the coronavirus disease 2019 (COVID-19) pandemic caused by severe acute respiratory syndrome coronavirus 2 (SARS-COV-2) has been intensifying. COVID-19 remains an ongoing global concern. Prevention is the best approach. Although COVID-19 vaccines block most SARS-COV-2 infections, vaccines are fighting a changing opponent. There is a need for methods of fast-acting prevention against evolving respiratory diseases.

Aerosolized pathogens are a leading cause of respiratory infection and transmission. A way to effectively inactivate the virus in the nasal/upper airway before it reaches the lungs would help prevent transmission and minimize disease symptoms in patients. To date, providing a pharmaceutical agent to the nasal cavity has been through a randomised injection of a pressurized product. The product is limited to either an aerosolized liquid or in powder form, with this process being rapid and arbitrary.

Apart from the unpleasant sensation of receiving such a high pressure injection, the injected agent is largely momentary. The metered volumes must be sufficient to ensure the user has sufficient of the product adhere to the nasal cavity wall. Give the randomness of this process, the metered volumes are based upon ideal conditions, including the user holding their breath, as inhalation or exhalation will disrupt the delivery process. Further, a high pressure injection of material can induce a sneeze reflex, ejecting all the injected material. The user then needs to consider whether they repeat the process, risking wasting material and exceeding the maximum metered dose. If the user does not repeat the process, insufficient material may be applied.

Further, there is no “scaling” of this type of applicator. If a sustained delivery is required, the only option is to repeat frequently, exacerbating the aforementioned problems each time the process is repeated.

### SUMMARY OF THE INVENTION

In a first aspect, the invention provides a device for providing an inhibition agent to a nasal cavity of a user, the device comprising: an insertion element, said insertion element having a surface having the inhibition agent; said insertion element arranged to insert the surface into the nasal cavity, such that the inhibition agent is placed to receive an inhalation and/or exhalation airflow.

The invention overcomes the problems of the prior art by providing a direct application, through physical placement, of the desired inhibition agent.

The physical placement may be in the form of:
i) A scaffold where the inhibition agent is a topical inhibitor on the scaffold that actively deactivates aerosolized pathogens. This may be in the form of the material selected for the scaffold or a composition placed on the surface of the scaffold;
ii) A scaffold where the inhibition agent is a pharmaceutical agent on a surface of the scaffold having a pharmaceutical release mechanism;
iii) Direct placement of the agent to the nasal cavity wall through topical, or direct transfer, rather than through the vagaries of an injected particle dosage;
iv) Physical embedment of a device having the pharmaceutical agent to the nasal cavity wall.

In each case, a precise volume of the inhibition product is applied, without risk of adverse conditions such as sneezing, or inhalation and exhalation. Further, the delivery of the agent is sustained, with each of the above mechanisms having a measurable and precise delivery of the agent, through use of a non-aerosolized liquid or a gel. Repeat applications are not required for sustained delivery, as is the case for the prior art.

### BRIEF DESCRIPTION OF THE DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIGS. 1A to 1C are various views of embodiments according to different aspects of the present invention;

FIGS. 2A to 2L are various views of a delivery device and packaging according to a several embodiments of the present invention;

FIGS. 3A to 3C are various views of a delivery device according to a further embodiment of the present invention;

FIGS. 4A and 4B are isometric detailed views of a delivery device according to a further embodiment of the present invention;

FIGS. 5A to 5C are various views of a delivery device according to a further embodiment of the present invention;

FIGS. 6A to 6H are various views of a delivery device and system according to a further embodiment of the present invention;

FIGS. 7A to 7C are elevation views of a delivery device and packaging according to a further embodiment of the present invention;

FIGS. 8A to 8F are sequential views of a delivery process according to a further embodiment of the present invention;

FIGS. 9A to 9D are sequential views of a delivery process according to a further embodiment of the present invention;

FIGS. 10A to 10E are sequential views of a delivery process according to a further embodiment of the present invention;

FIGS. 11A to 11C are various views of a delivery device according to a further embodiment of the present invention;

FIGS. 12A and 12B are various views of a delivery process according to a further embodiment of the present invention;

FIGS. 13A to 13D are various views of delivery devices according to several embodiments of the present invention;

FIGS. 14A to 14C are various views of delivery devices according to several embodiments of the present invention;

FIGS. 15A to 15E are various views of a delivery device according to twelfth embodiment of the present invention;

FIGS. 16A and 16B are elevation views of delivery processes according to two further embodiments of the present invention, and;

FIGS. 17A to 17D are various views of a delivery process according to a thirteenth embodiment of the present invention.

### DETAILED DESCRIPTION OF THE PREFERRED EMBODIMENTS

In general, the invention provides a nasal lining delivery device for delivering an inhibition agent to the nasal cavity for aerosol pathogen deactivation. The inhibition agent is directed to the deactivation of aerosol pathogens, with the inhibition agent being either (i) a pharmaceutical agent or (ii) a topical inhibitor positioned for pathogen contact.

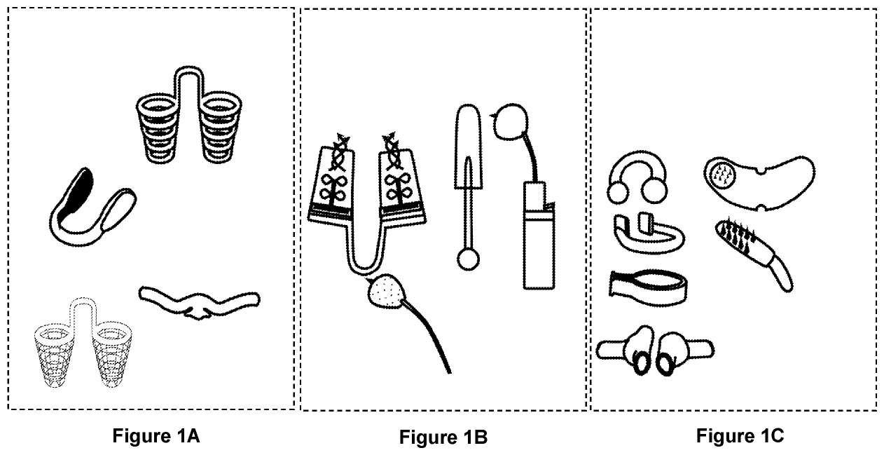

FIGS. 1A to 1C show various embodiments of the invention categorized under different aspects, but each demonstrating delivery through physical placement of the desired inhibition agent. The various aspects provide different forms of an insertion element with the inhibition agent applied onto a surface of the element. The insertion element is arranged to be inserted into the nasal cavity, such that the inhibition agent is placed so as to receive an inhalation and/or exhalation airflow.

In some aspects, the surface remains in the nasal cavity, whereas for other aspects the inhibition product is transferred to the nasal cavity wall, and the surface removed. In each aspect, however, the delivery of the agent avoids the randomness and momentary presence in the cavity, which is a characteristic of the prior art.

For instance, a first group 5 involves a filter deactivation system whereby the inhibition agent is a topical inhibitor either as the material used for the surface, or placed on the surface of a scaffold, with lobes of the scaffold representing insertion elements. A second group 10 involves the inhibition agent being a pharmaceutical agent, in the form of a deactivation coating on a surface of a device with the delivery mechanism being placement of the device in the nasal cavity, with a surface arranged to contact the nasal cavity wall to transfer the agent. A third group 15 involves the inhibition agent also being a pharmaceutical agent, comprising a device providing a sustained delivery of the pharmaceutical agent over a defined period.

For the first group 5, this aspect may have two functions:
a) Filter for the deactivation of airborne pathogens and;
b) Release of salt crystals to help thin mucus.

### (a) Deactivation of Airborne Pathogens with Salt-Based in Nasal Filters

Nasal inserts are designed to be inserted into the nasal cavity. The device actively “filters” the air and deactivates aerosolized pathogens as it enters the nasal cavity. Deactivation of aerosolized pathogens occurs due to interaction with the salt within/in/on the device. During inhalation and exhalation, the salt material dissolves and recrystallizes, capturing and destroying the pathogens (as high salt concentration disrupts the morphology or pathogens).

The presence of salt provides virus deactivation capabilities, where the viral infectivity upon contact with salt-coated structures causes physical destruction of the aerosol virus during recrystallization of coated salts. The natural inspiration and expiration of human breath (in addition to airflow enhancement in the device) work in tandem to change the salt concentration within the structure leading to effective recrystallization of salt.

The material for constructing the device may be (i) built from salt/minerals, (ii) functionalized with sodium chloride salt, calcium salt, and/or magnesium salt, (iii) impregnated or pre-blended with polymers, or (iv) core-shell structure with salt-based core fibre within.

A device according to this aspect may be manufactured as a:
i. Core-shell structure with salt-coated material loaded in the core, for example, polypropylene fibres coated with NaCl;
ii. Salt-based structure, i.e. embedding salt into polymeric structures
iii. Salt-coated surfaces
iv. 3D printed salt structure

The salt material may be sodium chloride, calcium, magnesium salts, ammonium sulphate, potassium chloride, Himalayan salt and/or potassium sulphate.

### (b) Delivery of Salt from the Device Matrix Helps to Thin Mucus

The other function for this aspect of the present invention is to deliver salt particles. Salt particles, when inhaled, draw water into the airway and lungs and thin out mucus. It also helps to make coughs more productive in COPD or cystic fibrosis patients with thick septum.

The delivery of salt droplets or exposure to salts clears SARS-COV-2 droplets from the upper airway. Moreover, the delivery of salt droplets or exposure to salts may lead to a reduction in symptoms of respiratory diseases in the population that had access to breathing humid and salty air.

The matrix material may include polycaprolactone (PCL), polyglycolide (PGA) polylactic acid (PLA), poly-L-lactic acid (PLLA), poly(lactic-co-glycolic acid) (PLGA), polyester and/or a blend of polymer materials

General steps to use a filter deactivation system according to the present invention:
1. Removal of the device from its packaging
2. Hold the “holder” segment of the device and direct the device towards the nostril
3. Using the “holder” segment to tilt and adjust, insert the device by physical adjustment
4. Device remains within the nasal space for between 30 mins to 8 hours depending on user preference and device type. The device is “activated” upon inhalation and exhalation.

The second group 10 includes devices having a formulation that inhibits viropexis.

SARS-COV-2 mainly spreads through respiratory droplets. The surface of SARS COV contains rod-like receptor binding domain (RBD) spikes that bind to human cells via the angiotensin-converting enzyme 2 (ACE2) receptor. Attachment and entry of SARS-COV and SARS-COV2 require the binding of the spike protein to the target receptor ACE2 on the cell surface. In this aspect, a device according to the present invention aims to deliver a formulation that inhibits viropexis by binding to the virus RBD and/or the ACE2 receptor in human cells.

Some actives that inhibit viropexis includes
Salvianolic acid [Binds both RBD and ACE receptor]
Dexamethasone [Binds both RBD and ACE receptor]
1,2,3,4,6-O-Pentagalloylglucose (PGG) [Binds to RBD]
Fluocinolone [Binds to RBD]
Iota-, lambda- and kappa-carrageenan
Azelastine, doxepin, chlorpheniramine, doxylamine, loratadine, desloratadine, and relevant antihistamines
Phenothiazines, thioridazine, trifluoperazine, and relevant antipsychotic drug

A layer of the active formulation is applied to the nasal cavity using the device. In one version of the device, a balloon is inflated to coat the nasal cavity. In another version, the in nose nasal devices are used. Upon contact with the formulation, the virus is irreversibly blocked and can no longer infect cells. This mechanism provides a physical barrier to respiratory viruses in the nasal cavity. The “blocked” viruses are then cleared naturally through the nasal mucus. Users can also use a saline wash to clear out the formulation when they are back home.

### FORMULATION EXAMPLES

### Example 1

Water, glycerine, propylene glycol, sodium hydroxide, preservatives, and stabilizers are formulated with the active.

### Example 2

Polyvinyl alcohol, propylene glycol, glycolic acid, ethylhexylgylcerin, preservatives, and stabilizers are formulated with the active.

(Preservatives/Stabilizer: Glycerol, trehalose, Methyl p-hydroxybenzoate, propyl p-hydroxybenzoate, aimidazolidinyl urea, and phenoxyethanol)

General steps to use a device under this aspect include:
1. Removal of the device from its packaging
2. Hold the “holder” segment of the device and insert the device into the nasal space;
3. Implementation may be through
   i. User to rotate device physically in the nasal space in rotation to coat the nasal space;
      Material is maintained in the nasal cavity, and then cleared when the user clears his/her nose.
      None of the material/fraction of particles are below 9 μm, as they are not intended for lung inhalation or residency.
   ii. Inflation of the device so that coating of active agents can be left behind in the nasal cavity;
   iii. Inflation of device (absorbent/weeping balloon) to “flood” the nasal cavity space
      Subsequently, the inhibition agents are directly delivered to the nasal space
      Inhibition agents can be delivered into the upper airway tract

OR Use as a regular spray

Layers may be coated onto the balloon either by spray coating, dip coating, and casting method. Apart from the material, the coating thickness and molecular weights of the material could also be adjusted to achieve the desired results.

### Materials for the Balloon

polyamide/polyether block copolymer (commonly referred to as PEBA or polyether block-amide), e.g. nylon 12
polyurethane
ethylene alpha-olefin polymer
thermoplastic polymers
elastomeric silicones, latexes, and urethanes
materials commonly involved in making balloon catheters

### Materials for Absorbent Material

Gelatin
Collagen
Hydroxylated PVA (polyvinyl-acetal) polymer
Hyaluronan polymers

The third group 20 is directed to the sustained delivery of inhibition agents from a temporary device.

This approach involves delivering pharmaceutically active agents, such as antibodies, vaccination, charged agents, or medication to a patient's nasal cavity. Delivering drugs directly into the nose and lungs reduces exposure to the rest of the body, potentially limiting side effects. Moreover, a sustained release without burst release may help provide a consistent supply without toxicity issues.

Device delivery of pharmaceutical agents via either (a) cartridge, (b) sublimation style gel (from solid to vapor state), (c) biodegradable membrane structure, (d) bioinert matrix structure, or (e) layer of coating on the membrane structure.

### Materials for Biodegradable, Biocompatible Film/Frameworks

PVA
PVA/PVOH
Starch-based biodegradable materials

### Materials for Non-Biodegradable, Biocompatible Frameworks

Polyacrylamide (PMA)
Poly(N-isopropylacrylamide) (PNIPAM)
Polyethylene terephthalate (PET)

### Materials for Gel Variation

Gelatin
Alginate
Collagen
Hydroxylated PVA (polyvinyl-acetal) polymer
Hyaluronan polymers
gelatin-alginate hydrogel

(with excipients such as PEG, salts, menthol salts, etc)

### Material for Adhesive Strip Device

Esterified hyaluronic acid (muco-adhesive gel)
Biocompatible adhesives

General steps to use a device under this aspect include:
1. Removal of the device from its packaging;
2. Hold the “holder” segment of
3. the device and direct the device towards the nostril;
4. Using the “holder” segment to tilt and adjust, insert the temporary anchorage of the device (physical-mechanical adjustment, pressing down of microneedles, and adhesive means), and;
5. Device remains within the nasal space for between 30 mins to 8 hours depending on user preference and device type.

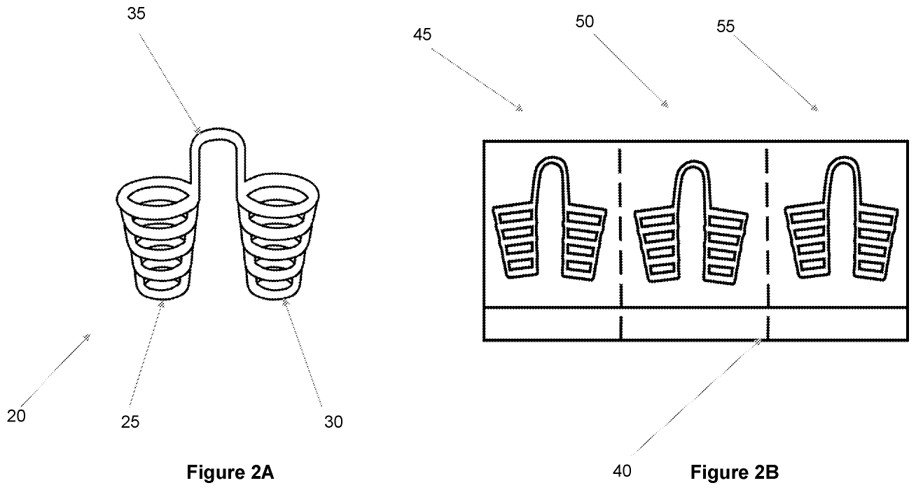

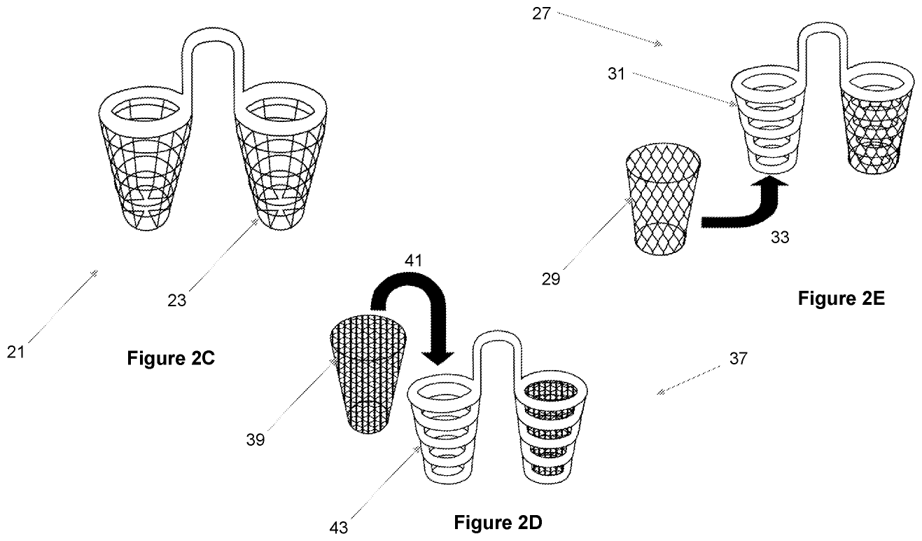

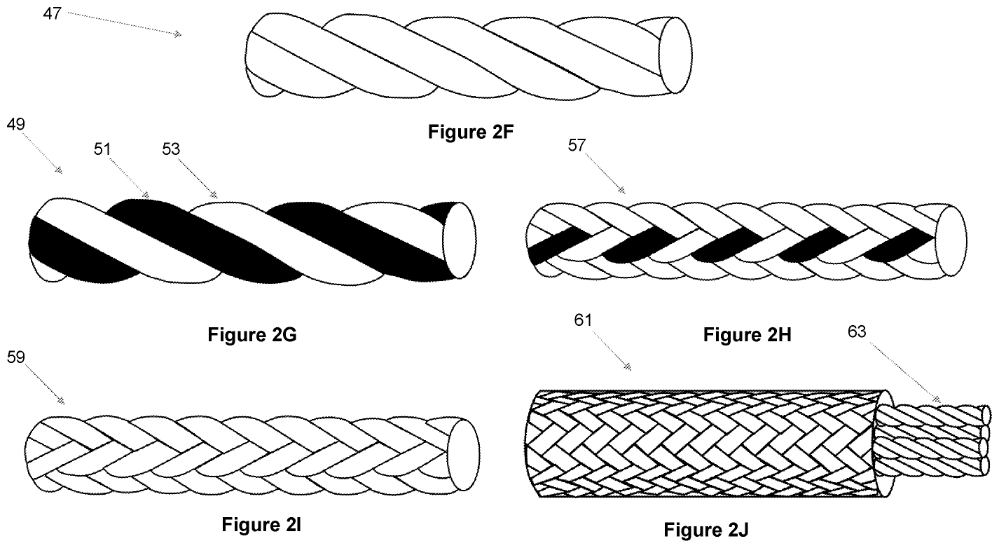

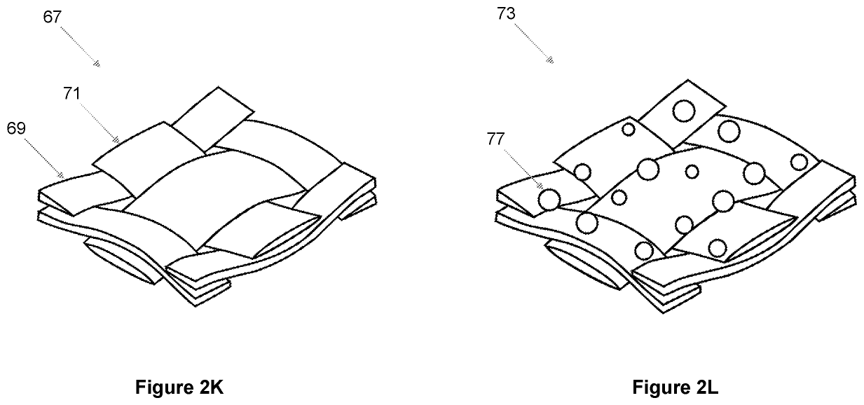

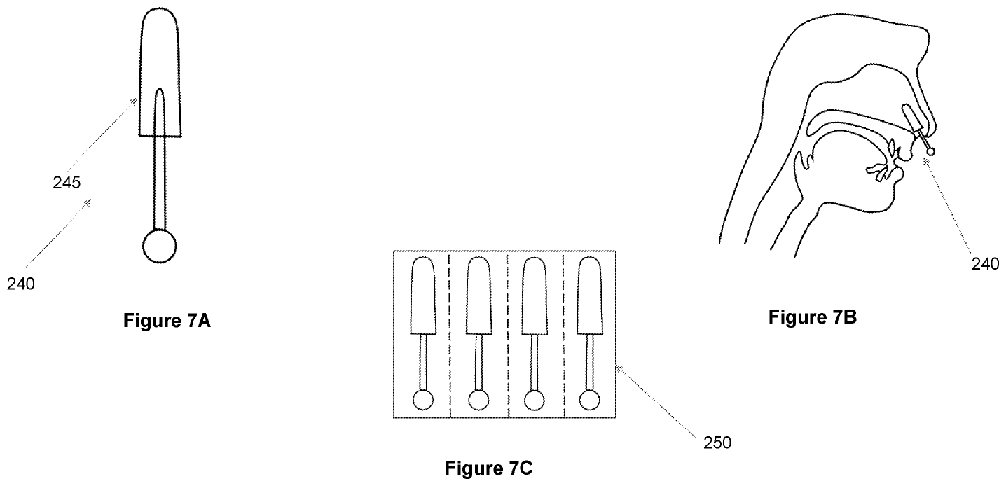

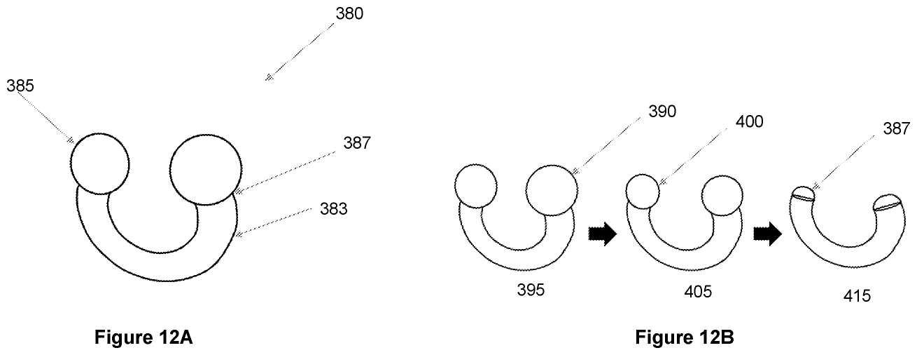

The following provides description of various examples falling within Groups 1, 2 and 3. Group 1 embodiments may be found in FIGS. 2-6, Group 2 embodiments may be found in FIGS. 7-11 and Group 3 embodiments may be found in FIGS. 12-17.

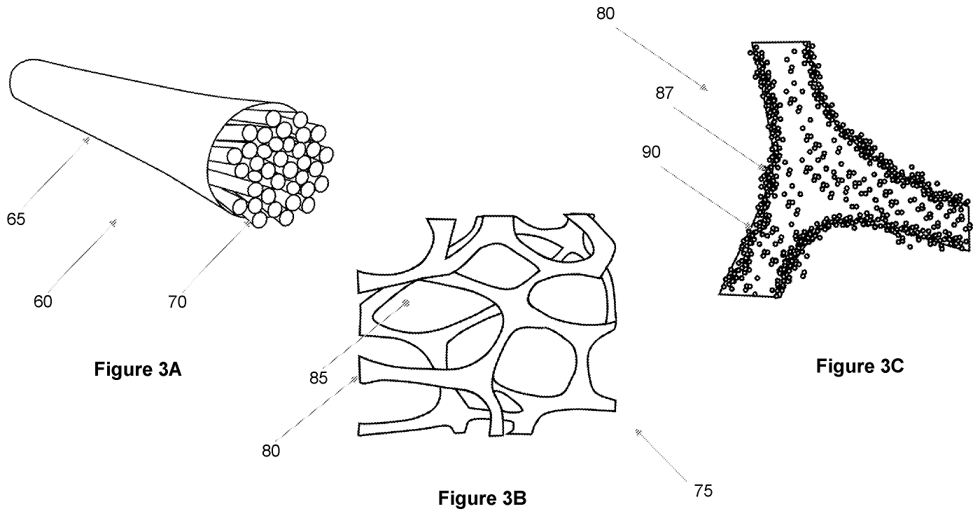

Turning to FIG. 2A, a device 20 is shown having two insertion elements, which in this case are lobes 25, 30, are respectively arranged to be inserted into the nasal cavity and connected through a bridge 35. The bridge 35 may be preloaded such that on insertion the device 20 is held in place through a spring action. The lobes include a topical inhibitor as the inhibition agent, which in this case is an impregnated salt crust as shown in FIGS. 3A to 3C. FIG. 2B shows a potential packaging 40 option whereby three of said devices 45, 50, 55 for use as required for the end user. The arrangements of FIGS. 2A to 2L may be used simultaneously with a mask. While a surgical mask is effective in suppressing respiratory virus droplets to an extent, side leakage has been commonly reported. The wearable version may help to reduce transmission commonly associated with smaller droplets of leaked viruses entering from the side, conferring greater inhalation protection. With these embodiments, when used concurrent with a surgical mask, may be especially beneficial in indoor areas with a high population density, poor ventilation, and hence a greater chance of virus exposure.

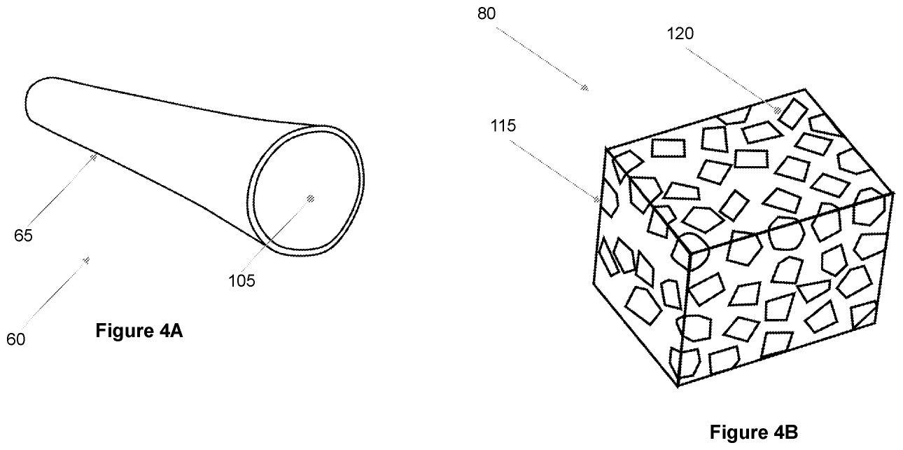

The lobes 25, 30 comprise an arrangement of filaments which, in this case, are substantially helical, defining a bore through which inhalation and exhalation by the user can pass. The actual arrangement of filaments may vary so as to provide sufficient surface area so as to impart the inhibiting agent. FIGS. 3A to 3C and 4A, 4B show detailed views of these filaments 60. The filament 60 includes an outer sheave 65 having a core material 70 of fibre structures or interconnected porous structures in a bore 105 of the outer sheave. FIG. 3B shows a close-up of the porous structure 75 having a matrix 80 separated by interstitial pores 85. FIG. 3C shows the matrix 80 in close-up whereby a surface 87 of the matrix 80 includes a layer of minerals, for instance, NaCl as a coating 90. The NaCl coating may have an extremely high concentration applied through immersion and drying or other appropriate mechanism for placing the mineral layer.

FIGS. 2C to 2E show alternative embodiments to that of FIG. 2A, where a scaffold having filaments is replaced with a scaffold 21 having by a mesh 23. The mesh 23 acts in the same way as the filaments by providing a surface on the mesh upon which the inhibition agent, in this case a topical inhibitor, but may be placed. Such a mesh may be beneficial in that a larger surface area of a finer mesh may be used. As with FIG. 2A, each of the scaffolds of FIGS. 2C to 2D may instead provide a surface for receiving a pharmaceutical agent rather than the topical inhibitor.

FIGS. 2D and 2E are further differentiated by having the mesh 29, 39 being selectively replaceable 33, 41. Thus the devices 27, 37 may provide several parts being a receiving part 31, 43 for receiving a mesh 29, 39 having the inhibition agent applied thereto. This may have several benefits including having the receiving parts unladen with the inhibition agent, and perhaps allowing the receiving part to be purchased separately, and the mesh being impregnated with several different inhibition agents. The end user may then select the inhibition agent they require by simply selecting the correct mesh. The mesh can then be discarded after use, and a replacement selected.

The embodiments of FIGS. 2D and 2E vary as to the connection with the receiving part 31, 43. In FIG. 2D, the mesh fits inside the lobe of the receiving part, and so protected from crushing on insertion into the nasal cavity. In FIG. 2E, the mesh fits over the lobe, with the lobe acting as a skeletal support to the mesh.

In a further embodiment, the inhibition agent may be the material used to form the mesh.

FIGS. 2F to 2J show variants which may be used for a device structure comprising a metallic material in the form of mesh or braided or as sleeve. The device may be braided 47, 49, 57, 59, 61 with material containing zinc, silver, magnesium, and copper, brass, copper-nickels, nickel alloy, copper alloy. Metal materials can be used as the core material 63 or blended 51, 53 as metal ions. Viral inhibition can happen through binding with viral proteins or disruption of viral capsid integrity upon contact.

FIGS. 2K and 2L show filter media 67, 73, which may be more breathable, aiding in the comfort of the user. Here, the nasal device may include a nanofibrous membrane made of a non-woven polypropylene substrate 69, 71. Other potential materials, including polymer electrets, polyethylene, polyesters, polyamides, polycarbonates, polyphenylene oxide, and polyester-rayon fibers could also be used for this filter membrane layer.

Zinc or silver nanoparticles 77 may also be added to increase the device's antiviral efficiency with low toxicity to humans.

As shown in FIGS. 4A and 4B, the outer material 65 may be composed of two immiscible materials such as a bio-compactable polymer, PCL 120 with NaCl crystals 115. Upon contact with moisture such as inhalation and exhalation of the user, the salt crystal may leach out into the nasal cavity and respiratory path helping to thin mucus and reduce congestion.

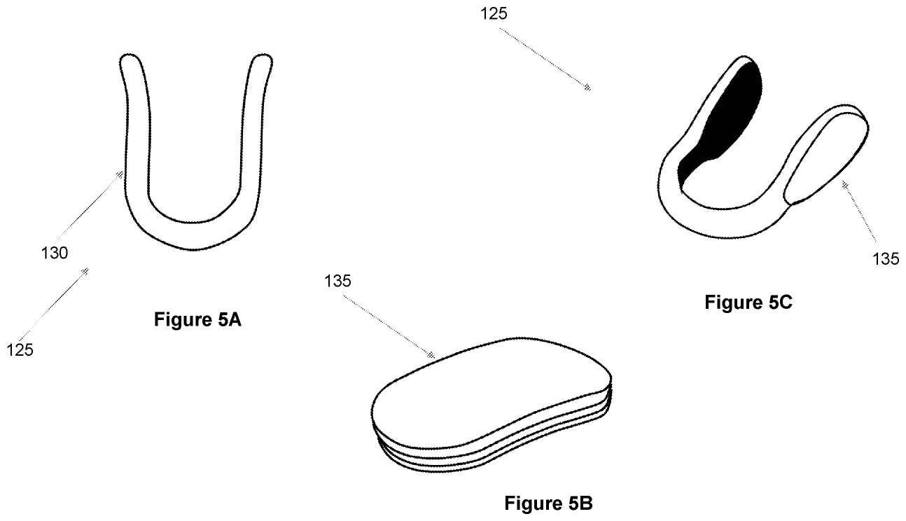

FIGS. 5A to 5C show a further Group 1 embodiment whereby a clip 125 is arranged to clamp on to the nasal septum. The clip 125 comprises a spring element 130 for providing the resilient clamp of the septum. The second component includes a surface having a filter 135 that traps and deactivates aerosol pathogens as they enter the nasal cavity.

Thus, by inserting the spring element 130 and clamping onto the septal the resilience of this device allows the securing of the device by pinching the U bend towards the nasal septum.

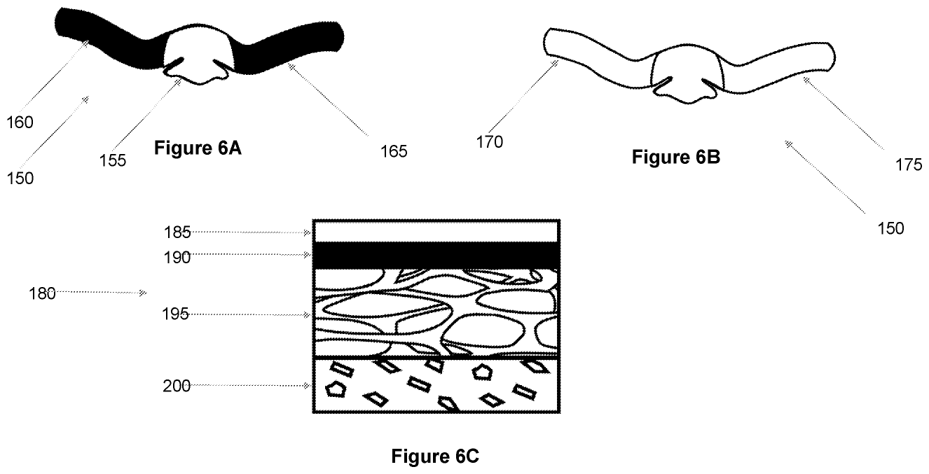

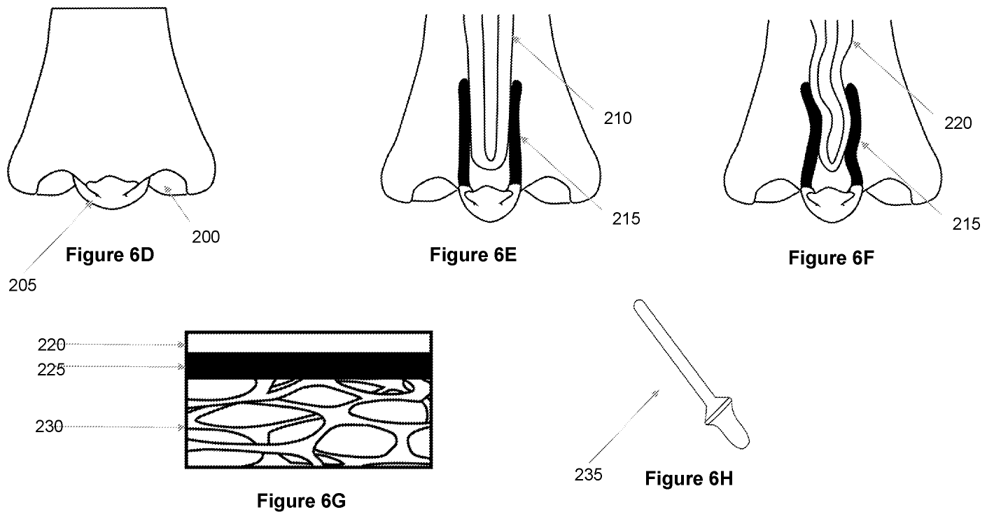

FIGS. 6A to 6F show a further embodiment of a Group 1 device. FIGS. 6A and 6B show a flexible nasal device suitable for users with a deviated or irregular nasal septum. The device 150 is shaped to accommodate the tip of the nose as well as ends or positioning into the nasal septum. The flexibility of the device allows for uses with deviated or irregular nasal septum, the flexibility of positioning the device 150.

It comprises a flexible polymeric strip 170, 175 placed about a central core 155 that allows users to adhere arms 160, 165 to the nasal septum via a bio-adhesive. FIGS. 6C and 6G show alternative arrangements of the layer structure. FIG. 6C shows a four-layer structure 180 with an adhesive layer 185, a support material 190, a filter layer 195 and a release layer 200 to help with nasal decongestion. FIG. 6G shows an alternative arrangement having only three layers (adhesive 220, support material 225, filter layer 230) such that the nasal decongestion layer is omitted. It will be appreciated that having merely an adhesive layer, supported material and filter layer is sufficient for many applications.

FIGS. 6D, 6E and 6F show the application of the flexible nasal device 205. Here, the device 205 is inserted into the nasal cavity 200 such that the flexible arms 215 grouped the nasal septum 210. By having both flexible arms 215 with an adhesive layer where the septum 220 is deviated, the flexibility of the arms permits adhesion to the septum to ensure a secure grip.

In a further embodiment, FIG. 6H shows an assistive nasal rod 235 to assist in the positioning of the nasal strip 205 toward the septum.

FIGS. 7A to 7C show an embodiment of the Group 2 devices. Here a device 240 including a delivery head 245 includes an applied layer of solid gel placed on a surface of a delivery head 245, with the delivery head acting as an insertion element. The delivery head 245 is inserted into the nasal cavity with the coating applied to the nasal cavity wall by moving the head in a rotating movement and consequently swapping and coating the nasal cavity.

Where the Group 1 devices maintain the surface—with the active agent—within the cavity, the Group 2 devices transfer the active agent to the nasal cavity wall, with the surface then being removed. In both cases, the active agent is placed to receive an inhalation and/or exhalation airflow.

The device 240 may be packaged 250 as a multi device blister pack to facilitate multiple uses. Once the solid gel has been applied, the user may blow their nose or use bottled saline to help wash or clear out the content once the gel has entrapped the pathogen particles.

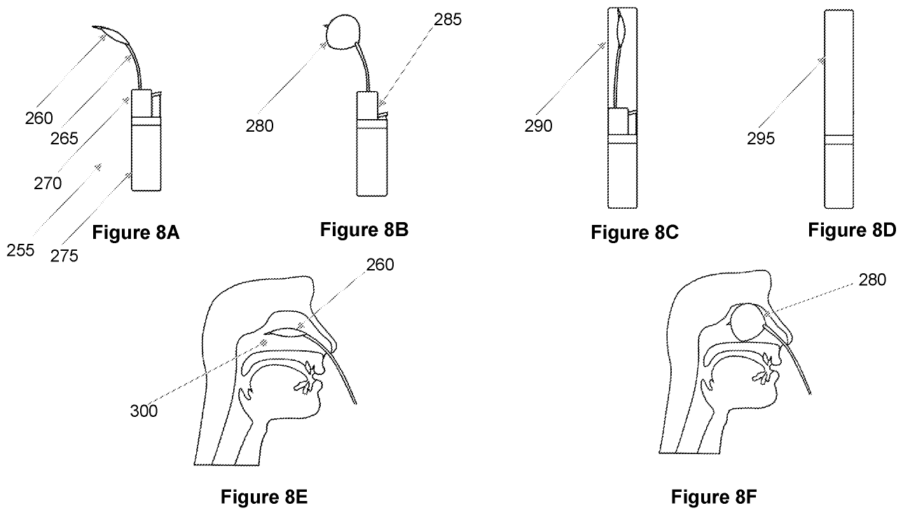

FIGS. 8A to 8F show a further embodiment of the Group 2 devices. In this embodiment a device 255 includes a deflated balloon 260 connected to a canister head 270 and canister 275 via an inflation tube 265. On triggering a switch 285, the canister provides air to inflate the balloon 280. Prior to use, the deflated balloon may be packaged with a protective cap 290, 295.

The balloon includes a surface having a coating of the inhibition agent. As the deflated balloon 260 is inserted into the nasal cavity 300, this corresponds to an insertion element. The resilience of the balloon surface allows the nasal cavity to be filled by the inflated balloon and thus applying the coating on the balloon surface to the walls of the nasal cavity. By holding the inflated balloon in place from several seconds up to minutes, transfer or delivery of the inhibition agent to the nasal cavity wall is achieved with a high degree of confidence. Given the nature of the coating transfer to the nasal cavity wall, the device 255 may provide further features including providing a detection or sensor for intra-nasal pressure, a sensor to guide or indicate the positioning within the nasal cavity of the inflated balloon or even a visual guide for the position of the balloon.

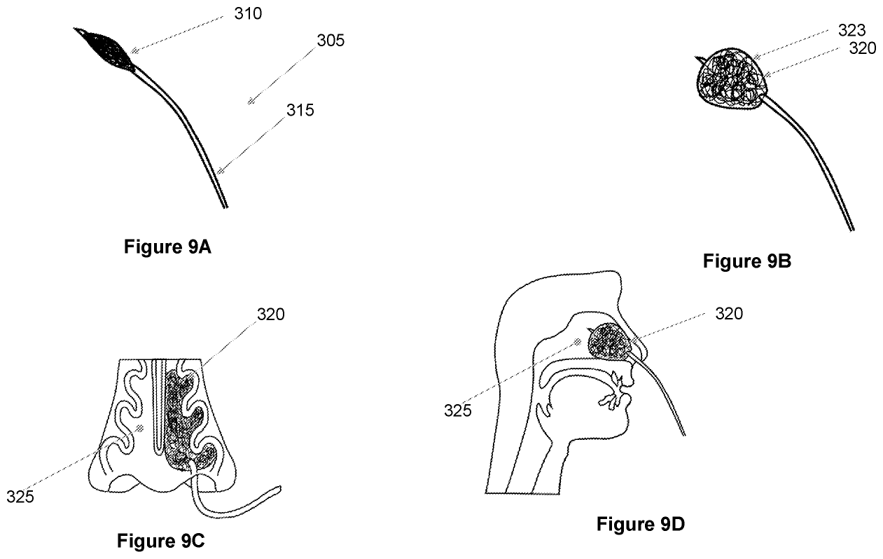

FIGS. 9A to 9D show a further embodiment similar to that of FIG. 8. Here a device 305 includes a deflated balloon 310 and an air supply 315. The air supply is arranged to inflate 320 the balloon whilst in the nasal cavity 325. In this embodiment, however, the balloon 320 includes a surface 323 having an absorbent material arranged to release the active inhibition agent on inflation. The process involves soaking the deflated balloon in a formulation, for the absorbent surface 323 to receive the active agent, then inserting the tip/insertion element with the soaked absorbent material into the nasal cavity 325. The user then coats the nasal cavity wall by moving the inflated balloon in a rotational movement within the cavity or holding the inflated balloon in the cavity for several seconds up to several minutes depending upon the requirements. The balloon can then be deflated and removed having delivered the inhibition agent to receive an inhalation and/or exhalation airflow.

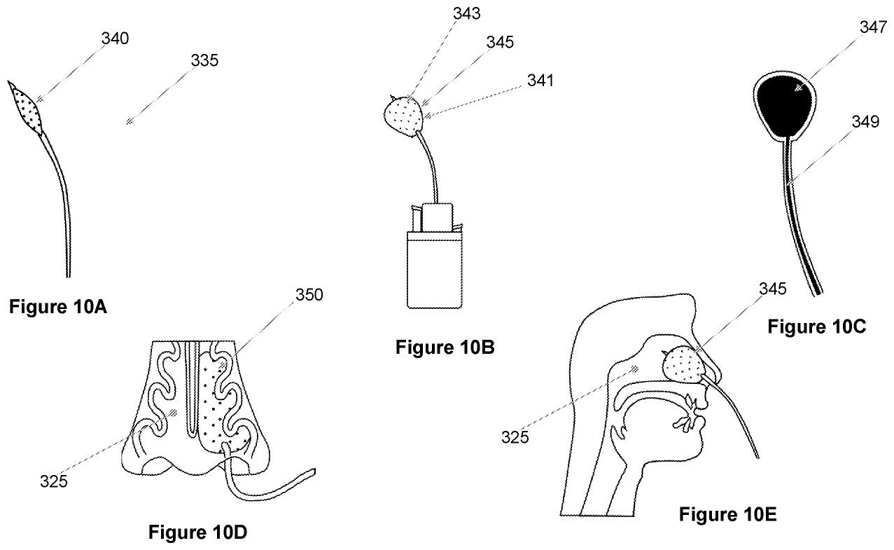

In a still further embodiment involving an inflation of a balloon, a FIGS. 10A to 10E show a similar device 335 having a deflated balloon 340. The balloon 345 is inflated in the nasal cavity 325 as with the previous embodiment. The difference in this case however, is that the balloon includes pores 343 in the surface 341 through which a solution 349 is directed from the internal chamber of the balloon 347. The active agent is then permitted to weep through the pores 345 onto the surface 341, so as to coat the nasal cavity wall. Thus, the balloon 345 is inflated 350 within the nasal cavity 325 and a solution is delivered to the nasal cavity wall through the weeping pores and surface. The balloon can then be deflated and removed.

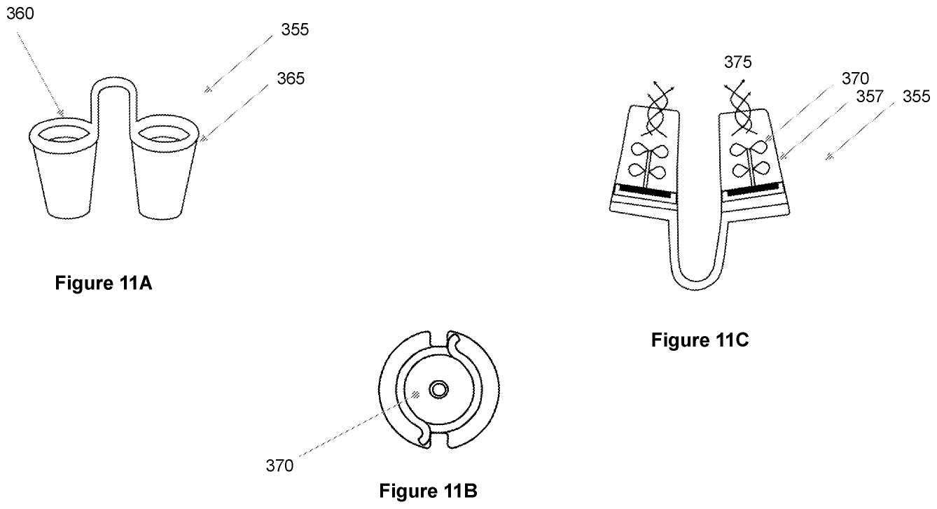

FIGS. 11A to 11C show a further embodiment of the group 2 devices. Here, an insertion device 355 similar to that of FIG. 2A includes insertion elements, or lobes 360, 365 connected by a bridge so as to insert into the nasal passages and clamp through a preloading of the resilient bridge. The device differs from that of FIG. 2A by providing a turbine device 370 within tubes 357 of the lobes 360, 365. As the user inhales, the inhalation drives the turbine 370 which push the inhibition product 375 located on internal surfaces within the lobes into the nasal cavity. Thus, with each lobe having a supply of the inhibition product, such as in a powder form, inhalation and subsequent driving of the turbines releasing the active product into the user. It will be appreciated that to avoid exhalation from directing the inhibition product outwards that the turbines are arranged to rotate in one direction only. Thus, on exhalation the turbines are not driven and thus the inhibition product is not entered to the atmosphere.

FIGS. 12A and 12B show one embodiment of the Group 3 devices. Here, a device 380 includes two components being a bridge 383 having ends acting as insertion elements. These elements are resiliently retained in the nasal passage and an active release component 385 positioned on a surface 387 of the bridge 383 is arranged to rest on both sides of the nasal septum. The active release component is arranged to provide sustained release of the active ingredient into the nasal cavity to interact with the user's inhalation and exhalation. FIG. 12B shows a progressive reduction in the active release component 390, 400 as the active ingredient is released, until the bridge surface 387 is empty of the active agent.

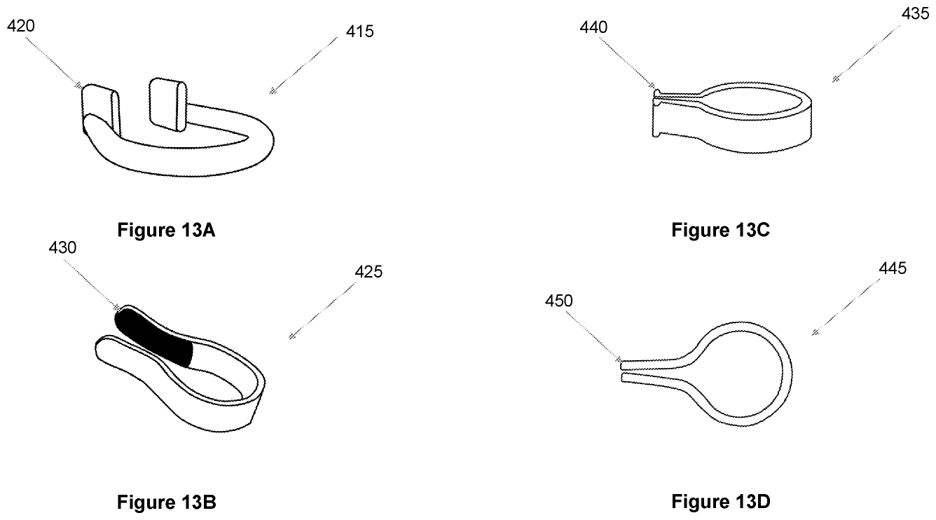

FIGS. 13A to 13D show various embodiments emulating that of the embodiment of FIG. 12. FIG. 13A shows a device 415 having a bridge connecting to the active release component 420.

FIG. 13B shows a device 425 whereby the active release component 430 on an internal surface of the device 425.

Similar arrangements are shown with FIGS. 13C and 13D having the devices 435, 445 with the coatings 440, 450.

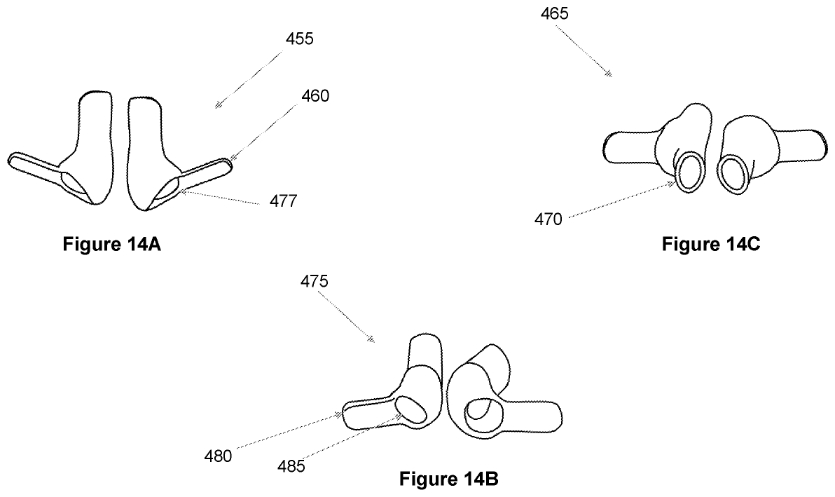

FIGS. 14A to 14C show anatomically fitted 460, 480 devices 455, 465, 475. Each of these embodiments are shaped to fit insertion elements into the nasal cavity so as to provide a conduit 477, 470, 485 to provide a path for inhalation and exhalation. The material of these embodiments is flexible so as to provide a resilient fit within the nasal cavity, allowing movement as the nose moves, and while providing sustained release of the active ingredient into the nasal cavity during inhalation and exhalation. In this case, the surface is within the conduit, and so having the active agent placed to receive an inhalation and/or exhalation airflow.

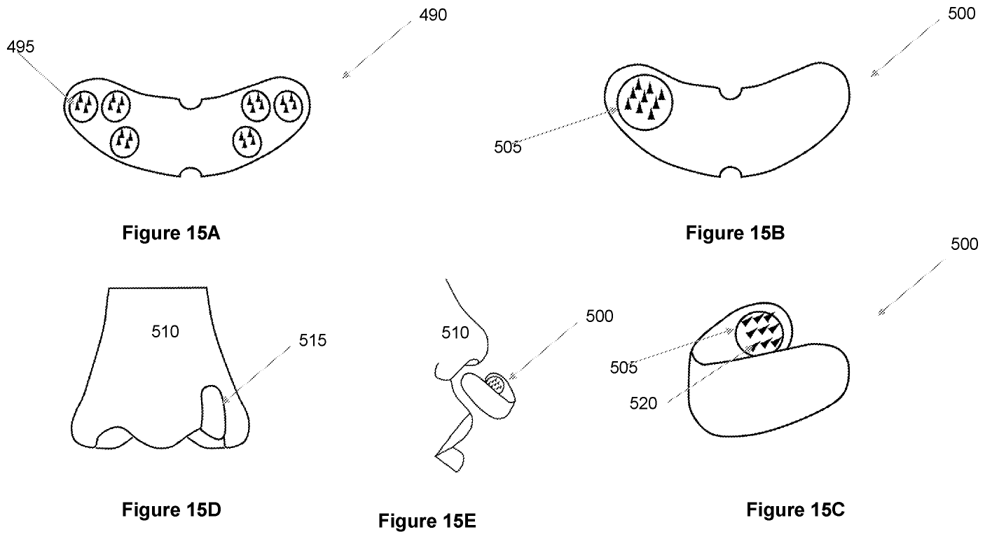

In a further class of the Group 3 devices, FIGS. 15, 16 and 17 provide a penetrating member approach for delivering the active ingredient.

In principle, the surface of the insertion element includes a penetrating member such as micro needles, which are inserted into the nasal cavity and firmly pressed against the wall from several seconds up to minutes. The device is left in place for the specified time before removing and disposing of the device.

In the embodiment of FIGS. 15A to 15E, a strip 490, 500 includes a surface having a micro needle patch 495, 505. The strip is bent into place so as to direct the insertion element, having the micro needle 520 patch 505 mounted thereto, into the nasal cavity wall. In the embodiment of FIG. 15 the strip 515 is arranged to have an adhesive layer. Alternatively, the device may be resiliently clamped to the wall of the nose 510 so as to stay in place during transfer or delivery of the active ingredient.

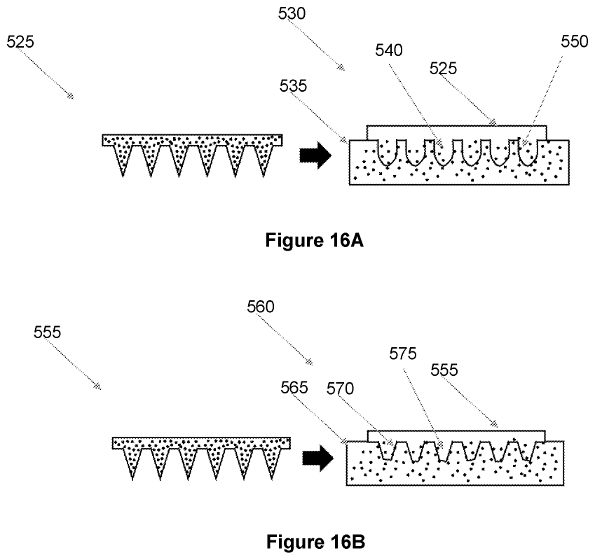

As shown in FIG. 16A, the micro needle patch 525 embeds 550 into the nasal cavity wall 535 so as to provide a delivery engagement 530. The active ingredient 540, which in this case is a hydrogel, penetrates the nasal cavity wall for the release of the active ingredient. Similarly for FIG. 16B, following the delivery engagement 560, the active ingredient embedded 570 in the micro needle patch 555 bonds with the nasal cavity wall 565 to impart the active ingredient 575, which in this case is a compact powder.

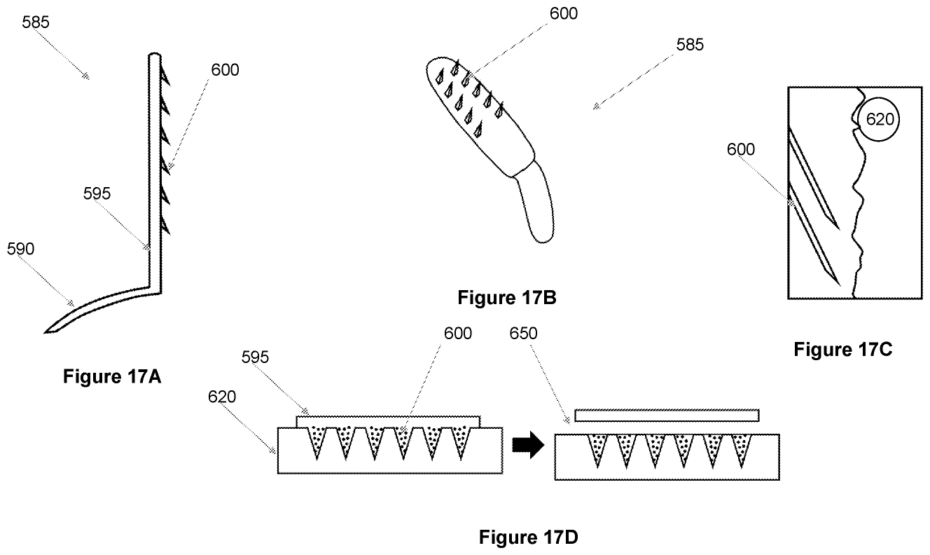

FIGS. 17A to 17D show an alternative micro needle embodiment whereby a device 585 includes a needle carrier 595 to which the micro needles 600 are attached and a tab 590 to assist in the placement of the device.

In this embodiment the micro needles 600 are detachable 650 such that on embedding the device 585 into the nasal cavity wall the needle support 595 is then withdrawn leaving the needles embedded in the cavity wall 620. The needles then progressively place or transfer the active ingredient without having to have the device in place. In a further embodiment the micro needles may be composed of the active ingredient such that the combination of the active ingredient and the micro needle allows for the sustained release of the active ingredient whilst the micro needles dissolve.

What is claimed is:

1. A device for providing an inhibition agent to a nasal cavity of a user, the device comprising:
an insertion element, said insertion element having a surface having the inhibition agent;
said insertion element arranged to insert the surface into the nasal cavity, such that the inhibition agent is placed to receive an inhalation and/or exhalation airflow.

2. The device according to claim 1, wherein the inhibition agent includes a pharmaceutical agent and/or a topical inhibitor.

3. The device according to claim 2, wherein the topical inhibitor is applied to the surface.

4. The device according to claim 2, wherein the surface is formed from a material, said material being the topical inhibitor.

5. The device according to claim 1, wherein the insertion element includes a scaffold of filaments, with the surface located on said filaments.

6. The device according to claim 1, wherein the insertion element includes a scaffold for receiving a mesh, with the surface located on said mesh.

7. The device according to claim 6, wherein said mesh is selectively replaceable on the said scaffold.

8. The device according to claim 5, wherein said filaments comprise an outer sheave and a core material of porous structures, said porous structures comprising a matrix separated by interstitial pores, said surface located on said matrix, said surface having a layer of inhibitor.

9. The device according to claim 8, wherein said matrix includes a mineral layer.

10. The device according to claim 8, wherein the insertion element includes spring element for resiliently engaging the nasal septum, the surface having a filter arranged to trap and deactivate aerosol pathogens.

11. The device according to claim 8, wherein the insertion element includes flexible arms for engaging the nasal septum, said arms having porous structures comprising a matrix separated by interstitial pores, said surface located on said matrix, said surface having a layer of inhibitor.

12. The device according to claim 1, wherein the insertion element is inflatable, with the surface located on said inflatable surface, said inflatable surface arranged to deposit the inhibition agent through contact with a wall of the nasal cavity wall.

13. The device according to claim 12, wherein the surface comprises an absorbent material for receiving the inhibition agent.

14. The device according to claim 12, wherein the inflatable insertion end is arranged to receive the inhibition agent into an internal chamber, said surface having pores arranged to weep the inhibition agent from the internal chamber onto the surface.

15. The device according to claim 1, wherein the insertion element includes a pair of tubes having a turbine in each, said turbines arranged to rotate on receiving an inhalation;
such that rotation of the turbines are arranged to drive inhibition agent from the surface inside said tubes into the nasal cavity.

16. The device according to claim 1, wherein the surface includes at least one penetrating member, said penetrating member arranged to penetrate a wall of the nasal cavity wall such that the surface is in contact with the wall and so embed the inhibition agent into the wall.
